(19) United States
(12) Patent Application Publication
(10) Pub. No.: US 2024/0058492 A1
(43) Pub. Date: Feb. 22, 2024

(54) NASAL ANTISEPTIC

(71) Applicant: PoviNez LLC, Philadelphia, PA (US)

(72) Inventors: JAVAD PARVIZI, Philadelphia, PA (US); Michael YAYAC, Philadelphia, PA (US); EMANUELE CHISARI, Philadelphia, PA (US); HARRY BRITTAIN, Philadelphia, PA (US); JEONGEUN CHO, Philadelphia, PA (US); MAYANK PARMAR, Philadelphia, PA (US)

(21) Appl. No.: 18/386,831

(22) Filed: Nov. 3, 2023

Related U.S. Application Data
(63) Continuation of application No. PCT/US2022/029858, filed on May 18, 2022.
(60) Provisional application No. 63/190,041, filed on May 18, 2021.

(51) Int. Cl. A61L 2/18

(52) U.S. Cl. CPC A61L 2/18; A61L 2101/50

(57) ABSTRACT

An antimicrobial composition comprising: (1) povidone iodine (PVP-I) in a concentration of 5 to 10% w/w of the composition; and (2) carboxymethylcellulose (CMC) in a concentration of 0.1 to 5 % w/w of the composition.

### REFERENCE TO RELATED APPLICATION

This application is based on U.S. Provisional Application No. 63/190,041, filed May 18, 2021, which is hereby incorporated by reference.

### FIELD OF INVENTION

The present invention is directed generally to a topical composition for nasal application, and, more specifically, to a nasal antiseptic comprising povidone-iodine.

### BACKGROUND

Surgical site infections (SSIs) are infections of the incisions, organs, or internal spaces that occur after surgery. The prevention of SSIs is increasingly important as the number of surgical procedures performed in the United States continues to rise. Public reporting of process, outcome, and other quality improvement measures is now required, and reimbursements for treating SSIs are being reduced or denied. It has been estimated that approximately half of SSIs are preventable.

Based on Center for Disease Control (CDC) 2017 guidelines, bioburden reduction prior to surgery is critical. Numerous studies and historical information support the fact that the surgical site infection and hospital acquired infections can be prevented by wide use of effective antimicrobial products. Thus, the CDC recommends that patients should shower or bathe (full body) with soap (antimicrobial or non-antimicrobial) or use an antiseptic agent on at least the night before the operative day. Numerous studies have shown that application of an antibacterial agent to the nares prior to surgery also reduces bioburden and may reduce the incidence of SSIs.

There is an obvious need for an effective product with wide chemical and physical properties that can allow the prevention protocol to be conducted in the most effective way. Measures such as hand hygiene require antiseptics that have been dispersed in the form of a colloidal solution. Preoperative bathing might require either colloidal or solid antiseptic strategies for use on the body, while nasal decolonization requires solutions that can be applied using a swab or of a spray. A similar scenario with many possible strategies, delivery mechanisms, and solutions requires the development of approaches that allow for the multiple physical properties associated with every antiseptic used. In particular, the application to the nostrils faces multiple challenges to improve both efficacy and delivery of the antimicrobial product, such as long-lasting adhesion to the inner surface of the nostril to allow for longer exposure time of the biotic product.

There are many anti-microbial agents on the market which can potentially be used to combat SSIs. One such drug is polyvinylpyrrolidone complexed with iodine (also known as povidone-iodine (PVP-I)), a widely available antiseptic that is commonly used in clinical settings, including for skin disinfection before and after surgery. Since the iodine is tightly complexed, germicidal properties can be obtained without the toxicity or staining concerns associated with compositions containing elemental iodine.

For nasal applications, the viscosity of a PVP-I formulation is an important factor is its efficacy. Application of PVP-I to the nares, for the purpose of reducing bioburden, is associated with “low period of contact” as the liquid agent tends to drip out of the nose and does not remain in the nares long enough to kill resident bacteria. Application of solid agents to the nares, like mupirocin ointment, is also problematic. For an anti-bacterial agent to be effective it needs to be applied to the upper (deep) region of the nares. Application of solid agents to the upper part of nares requires an uncomfortable applicator.

Thus, Applicant has identified the need for a PVP-I formulation that has the range of physical properties that optimizes application of the formulation to all parts of the nares, including external and deeper portions of the nasal cavity, and allows for the product to remain in contact with the nasal mucosa long enough to have antimicrobial effects. The present invention fulfils this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

One aspect of the present invention is a povidone-iodine (PVP-I) formulation that contains carboxymethylcellulose to alter the physical and chemical properties of the formulation, and, in particular, to increase the viscosity of the formulation to overcome the aforesaid problems, without altering the efficacy of the PVP-I (e.g., PVP-I's preservative, antimicrobial, antiseptic, antifungal, antibacterial, and/or virucidal properties). Specifically, Applicant has found that adding carboxymethylcellulose (CMC) to a nasal antiseptic comprising povidone-iodine with carboxymethylcellulose (CMC) increases the viscosity of the formulation greater than one would have expected. Without being bound by a particular theory, Applicant believes this unexpected result is due to the CMC forming CMC-I compounds with the PVP-I, thereby causing the composition to be anisotropic.

In one embodiment, the present invention relates to an antimicrobial composition comprising: (1) a povidone iodine (PVP-I) in a concentration of 5 to 10% w/w of the composition; (2) carboxymethylcellulose (CMC) in a concentration of 0.1 to 5% w/w of the composition.

In one embodiment, the present invention relates to an antimicrobial composition comprising: (1) povidone iodine (PVP-I); and (2) carboxymethylcellulose (CMC) in a concentration sufficient such that CMC interacts with the PVP-I to form CMC-I, thereby causing the composition to be anisotropic.

In one embodiment, the present invention relates to a method of making the aforementioned composition wherein PVP-I is mixed dry with CMC to increase solubility of the product.

In one embodiment, the present invention relates to a method of making the aforementioned composition wherein PVP-I is not exposed to temperatures above 60° C.

In one embodiment, the present invention relates to using the aforementioned composition comprising: applying the composition to the nares of a patient prior to performing a medical procedure.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

The present invention describes the use of carboxymethylcellulose (CMC) as excipient to adjust and tailor the physical and chemical properties of PVP-I nasal antiseptic, such as its viscosity, without interfering with the efficacy of the PVP-I. Multiple combinations of other excipients can be used as adjuvant. Additionally, other ingredients, such as short- mid- and long-chain alcohols, benzalkonium chloride, chlorhexidine, or peroxides, can be used as active ingredients in combination with PVP-I to enhance its antiseptic and antimicrobial activity.

In one embodiment, the formulation has at least (1) povidone iodine or a combination of povidone iodine and another active ingredient, and (2) carboxymethylcellulose. In one embodiment, the formulation also comprises (3) a pH buffer, and (4) one or more surfactants. In one embodiment, the formulation also comprises a fragrance or essential oil. Still other additives will be obvious to those of skill in light of this disclosure.

Povidone iodine is an iodophor solution containing a water-soluble complex of iodine and polyvinylpyrrolidone (PVP) with broad microbicidal activity. Free iodine, slowly liberated from the polyvinylpyrrolidone iodine (PVP-I) complex in solution, destroys the bacterial membranes through iodination of lipids and oxidation of other compound present in both Gram-positive and Gram-negative organisms. Due to its unique characteristics, this agent is the active ingredient of multiple antiseptic solutions with a wide variety of applications. When solubilized in an aqueous solution, povidone iodine shows a viscosity equivalent to water, which allows its use as topical antiseptic for the skin and surfaces. However, in order to allow a long-lasting delivery of antimicrobial iodine and allow the killing of bacteria that resides in biological niche (e.g. Cutibacterium acnes or Staphylococcus aureus), some applications might require a lasting permanence at the site of application, or a longer exposure to the compound. This particular function can be tailored by changing the physical and chemical properties of the solution through the use of suitable excipients. The concentration of PVP-I in the antiseptic composition can vary by application, and one skill the art, in light of this disclosure, will be able to determine an optimal concentration without undue experimentation. In one embodiment, the concentration of PVP-I in the antiseptic composition is 0.1 to 20% w/w, and, in another embodiment, is 1 to 10% w/w, and, in another embodiment, is 3 to 15% w/w, and, in another embodiment, is 5 to 10% w/w. In one embodiment, the concentration of PVP-I in the antiseptic composition is at least 0.1% w/w, or at least 0.5% w/w, or at least 1% w/w, or at least 2% w/w, or at least 3% w/w, or at least 4% w/w, or at least 5% w/w. In one embodiment, the concentration of PVP-I in the antiseptic composition is no greater than 20% w/w, or no greater than 15% w/w, or no greater than 10% w/w, or no greater than 8% w/w, or no greater than 7% w/w, or no greater than 6% w/w, or no greater than 5% w/w.

When seeking to increase the viscosity of povidone-iodine solutions, Applicant researched multiple viscosity enhancers or thickeners. Examples of thickeners include alginic acid (E400), sodium alginate (E401), potassium alginate (E402), ammonium alginate (E403), calcium alginate (E404), agar (E406), carrageenan (E407), locust bean gum (E410), pectin (E440), gelatin (E441), silicon dioxide, polyacrylic acid, polyethylene glycol, castor oil, polyvinyl alcohol, polyurethanes, sulfonates, and cellulosics such as methyl cellulose, CMC, hydroxyethyl cellulose, and hydroxypropyl methylcellulose. However, most of these viscosity enhancers or thickeners failed to achieve the viscosity needed to avoid a long-lasting adhesion to nostril inner surface. An example of candidates tested is reported in Table 1.

TABLE 1

| | Candidate | Candidate | Candidate | Candidate | Candidate | Candidate | Candidate |
|---|---|---|---|---|---|---|---|
| Ingredient | 1 | 2 | 3 | 4 | 5 | 6 | 7 |
| w/v | Qty. | Qty. | Qty. | Qty. | Qty. | Qty. | Qty. |
| Povidone | 1-10% | 1-10% | 1-10% | 1-10% | 1-10% | 1-10% | 1-10% |
| iodine | | | | | | | |
| PEG3350 | 1-5% | 1-5% | | | 5% | | |
| PEG8000 | | | 1-5% | 1-5% | | | |
| Glycerin | 1% | 2% | 1% | 2% | 2% | | |
| alginate | | | | | 1-3% | | |
| silicon | | | | | | 1-3% | |
| dioxide | | | | | | | |
| pectin | | | | | | | 1-3% |
| Viscosity | 400-1200 | 450-1125 | 700-1600 | 700-1600 | 63-950 | 50-600 | 15-precipitation |
| (cP)* | | | | | | | (1.5%) |

*All measurements were taken at 25 ± 1° C.

Applicant focused instead on a cellulosic compound as a viscosity enhancer, finding that the cellulosic compound provided a greater than expected viscosity increase. For example, in a concentration of about 2% to 4% w/w resulted in a viscosity range of 1500 to 4130 cP (measured at 25±1° C.). This was found to be effective for Applicant's objectives. Without being bound by a particular theory, Applicant believes the CMC forms CMC-I compounds with the PVP-I, causing the composition to be anisotropic.

The viscosity enhancer that showed the best performance for a wide array of application was carboxymethylcellulose Sodium (CMC-Na). The use of Na-CMC allowed for the greater viscosity enhancement regardless of the ingredients used and the salt present in the solution.

Carboxymethylcellulose (CMC) or cellulose gum is a cellulose derivative with carboxymethyl groups (—CH2-COOH) bound to some of the hydroxyl groups of the glucopyranose monomers that make up the cellulose backbone. For solubility reasons, CMC is normally used as its sodium salt, being termed carboxymethyl cellulose sodium (CMC-Na). CMC-Na is commercially available, for example, Texturecel from DuPont.

The concentration of CMC in the antiseptic composition can vary by application, and one skill the art, in light of this disclosure, will be able to determine an optimal concentration without undue experimentation. In one embodiment, the concentration of CMC in the antiseptic composition is 0.01 to 20% w/w, and, in another embodiment, is 0.05 to 10% w/w, and, in another embodiment, is 0.1 to 5% w/w, and, in another embodiment, is 5 to 10% w/w. In one embodiment, the concentration of CMC in the antiseptic composition is at least 0.01% w/w, or at least 0.05% w/w, or at least 0.1% w/w, or at least 1% w/w, or at least 2% w/w, or at least 3% w/w, or at least 4% w/w. In one embodiment, the concentration of CMC in the antiseptic composition is no greater than 20% w/w, or no greater than 15% w/w, or no greater than 10% w/w, or no greater than 8% w/w, or no greater than 7% w/w, or no greater than 6% w/w, or no greater than 5% w/w.

In one embodiment, the viscosity of composition is at least 1500 cP, or at least 1800 cP, or at least 2000 cP, or at least 2200 cP, or at least 2400 cP, or at least 2600 cP, or at least 2800 cP, or at least 3000 cP, or at least 3400 cP, or at least 3600 cP, or at least 3800 cP, or at least 4000 cP. In one embodiment, the composition has a viscosity such that the composition remains in contact with the nares for at least 30 seconds, or at least 1 min, or at least 2 min, or at least 5 min, or at least 10 min.

In one embodiment, the other ingredients to improve stability, application comfort, and pH regulation of the antiseptic formulation. Most of these ingredients are salts, which can interact with the levels of viscosity.

For formulations comprising a combination of hydrophobic and hydrophilic substances, obtaining a homogenous mixture may be more readily achieved by preparing the composition as an emulsion. Inclusion of a surfactant to the present invention can increase the stability of the prepared emulsion. In comparison to cationic, anionic, and zwitterionic surfactants, nonionic surfactants demonstrate the least cytotoxicity and risk of skin irritation. Classes of nonionic surfactants include, for example, fatty alcohol ethoxylates, alkylphenol ethoxylates, fatty acid ethoxylates, ethoxylated amines, fatty acid amides, terminally blocked ethoxylates, fatty acid esters of glycerol, fatty acid esters of sorbitol, fatty acid esters of sucrose, alkyl polyglucosides. In one embodiment, examples of the surfactants include ppg-5-ceteth-10 phosphate, polysorbates, Lauramidopropylamine oxide, glycerine, dimethicone, polyethylene glycol (PEG) of any molecular weight, Nonoxynol-10, Silicon Dioxide, and vitamin E TPGS. In one embodiment, the concentration of one or more nonionic surfactants in the formulation ranges from about 0.5% to about 5% w/w.

In one embodiment, the formulation includes the addition of a pH buffering system, which is useful to maintain the chemical stability of the compound. More specifically, while CMC tends to be stable over a pH range of about 2 to 13, the use of a pH buffer system enhances the compounds short- and long-term stability. Multiple options exist depending on the desired pH of the formulation, such as buffers based on systems derived from malic acid, phosphoric acid, citric acid, lactic acid, boric acid, acetic acid, imidazole (glyoxaline), and carbonate/bicarbonate. Those of skill in the art will be able to determine, in light of this disclosure, without undue experimentation, the concentration of a pH buffering system to enhance short-term and long-term stability. In one embodiment, the concentration of a pH buffer is 0.01% to 5% w/w.

In one embodiment, fragrances are added to mask any unpleasant odors associated with other ingredients, and thus enhance the application of the formulation to the nasal area, and provide a more pleasant experience for the user. Fragrances that can be used include, for example, Geraniol, Diphenyl ether, Ethyl methylphenylglycidate, hexadecanoic acid, ethyl ester, m-Cresol, p-Cresol, menthol, and eucalyptol. Additionally, a wide variety of essential oils can be used. Essential oils are a mixture of aromatic volatile oils extracted from plant sources that are commonly used to impart fragrance to topical products. In addition to a pleasant odor, many essential oils exhibit antimicrobial properties, with current evidence suggesting eucalyptus oil has significant antimicrobial activity. In one embodiment, the fragrance is citrus oil or lavender oil or eucalyptus oil. In one embodiment, the fragrances has a concentration in the formulation of about 0.1% to about 1% w/w.

Additionally, the formulation can be safely mixed with other ingredients such as water, ethyl alcohol, isopropyl alcohol, quaternary alcohols, hydrogen peroxide, benzalkonium chloride, chlorhexidine gluconate, bacitracin zinc, neomycin sulfate, lidocaine hydrochloride, polymyxin B sulfate, or longer chain organic solvents. In one embodiment, the antiseptic composition is a solution. In one embodiment, the solvent for the antiseptic solution is water.

In one embodiment, the antiseptic composition is prepared by not exposing PVP-I to temperatures above 60° C. In one embodiment, the antiseptic composition is irradiated or otherwise treated to assure sterilization of the product for any indication or use.

### Example 1

To produce 100 g of antiseptic gel, a solution of preferred surfactant and 70 mL of purified sterile water is heated until reaching a temperature of 40° C. Then, the desired amount of PVP-I (ranging from 0.01 g to 20 g) and CMC (ranging from 0.1 g up to 5 g) are mixed as dry powders. After this, the mixed powders are slowly added to the water, while mechanical stirring and constant temperature (40° C.) is maintained. The process can take up to 1-2 hours to achieve complete solubility, whereupon the composition is allowed to cool down to room temperature. In the meantime, a chosen buffer is selected, in this case made of sodium citrate and citric acid. The buffer (s) should be selected to maintain a specific pH of the gel for a longer time, but buffers prepared with specific ratios tend to work better. In this case, with a targeted pH between 3.0 and 6.5, a range of 0.30 to 0.845 g of citric acid and a range of 1.0 to 2.1 g of sodium citrate are added to beakers, each of which contains 30 mL of purified water at room temperature. Once both solutions are ready, they can be mixed together. A high shear mixer or similar equipment can be used at this stage to ensure a homogenous emulsion is achieved.

### Example 2

To produce 100 mL of antiseptic gel, a solution of preferred surfactant and 70 mL of deionized sterile water is heated until reaching a temperature of 40° C. Then, the desired amount of PVP-I (ranging from 0.01 g to 20 g) is measured and slowly added, while mechanical stirring and constant temperature (40° C.) are maintained. The process can take up to 30 minutes to achieve complete solubility, whereupon the composition is allowed to cool down to room temperature. Then CMC (ranging from 0.1 g up to 5 g) can be weighted and prepared as dry powder. In the meantime, a chosen buffer is selected, in this case made of sodium citrate and citric acid. In this case, with a targeted pH between 3.0 and 6.5, a range of 0.30 to 0.845 g of citric acid and a range of 1.0 to 2.1 g of sodium citrate can each be added to beakers, each of which contains 30 mL of purified water at room temperature. Once both solutions are ready, they can be mixed together. CMC can now be added in solution with PVP-I and water slowly while mechanical stirring. This process will avoid exposure of PVP-I to high temperature that might affect its stability in the long term. Once both solutions are ready, they can be mixed together. A high shear mixer or homogenizer can be used at this stage to ensure a homogenous emulsion is achieved.

These and other advantages maybe realized in accordance with the specific embodiments described as well as other variations. It is to be understood that the above description is intended to be illustrative, and not restrictive. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled.

What is claimed is:

1. An antimicrobial composition comprising:
povidone iodine (PVP-I) in a concentration of 5 to 10% w/w of the composition;
carboxymethylcellulose (CMC) in a concentration of 0.1 to 5% w/w of the composition.

2. The composition of claim 1, wherein the concentration of CMC is sufficient such that CMC interacts with the PVP-I to form CMC-I, thereby causing the composition to be anisotropic.

3. The composition of claim 1 wherein the CMC is Na-CMC, which interacts with salts present in the solution without decreasing the viscosity of the final product.

4. The composition of claim 1, further comprising a pH buffer.

5. The composition of claim 4, wherein the pH buffer is citrate-citric acid.

6. The composition of claim 5, further comprising a surfactant.

7. The composition of claim 6, wherein the surfactant is polysorbate-80 in an amount between 0.01 to 4% w/w.

8. The composition of claim 1, further comprising a surfactant.

9. The composition of claim 7, wherein the surfactant is polysorbate-80 in an amount between 0.01 to 4% w/w.

10. The composition of claim 1, further comprising a fragrance or an essential oil from about 0.1 to about 5% w/w.

11. The composition of claim 1, further comprising water in a majority % w/w.

12. A method of making the composition of claim 1, wherein PVP-I is mixed dry with CMC to increase solubility of the product.

13. A method of making the composition of claim 1, wherein PVP-I is not exposed to temperatures above 60° C.

14. A method of ultimately optionally irradiating or otherwise treated to assure sterilization of the product for any indication or use.

15. A method of using the composition of claim 1 comprising:
applying the composition to the nares of a patient prior to performing a medical procedure.

16. The method of claim 15, wherein the composition has a viscosity such that the composition remains in contact with the nares for at least 30 seconds.

17. An antimicrobial composition comprising:
povidone iodine (PVP-I); and
carboxymethylcellulose (CMC) in a concentration sufficient such that CMC interacts with the PVP-I to form CMC-I, thereby causing the composition to be anisotropic.

18. The composition of claim 17, wherein the PVP-I has a concentration of 5 to 10% w/w of the composition, and the CMC has a concentration of 0.1 to 5% w/w of the composition.
